(19) United States
(12) Patent Application Publication
(10) Pub. No.: US 2008/0069908 A1
(43) Pub. Date: Mar. 20, 2008

(54) Compositions for safe and effective regression of dermal vessel tortuosity in psoriatic lesions

(75) Inventors: N.B. Baktha Reddy, Chennai (IN); Senthil Kumar, Trichy (IN); Vilambi NRK Reddy, Trichy (IN); Anil Torgalkar, Cranbury, NJ (US); N.R. Murugan, Trichy (IN)

Correspondence Address: BREGEN TECHNICAL CONSULTANTS L.L.C., 154 OLD CLINTON ROAD, FLEMINGTON, NJ, 08822, US

(73) Assignee: Apptec Inc., Cranbury, NJ (US)

(21) Appl. No.: 11/890,160

(22) Filed: Aug. 3, 2007

Related U.S. Application Data
(62) Division of application No. 11/288,923, filed on Nov. 28, 2005.

(51) Int. Cl. A61K 36/889; A61K 36/00; A61P 17/00

(52) U.S. Cl. 424/727; 424/725

(57) ABSTRACT

Topical compositions for Safe and Effective regression of Dermal Vessel Tortuosity in Psoriatic Lesions in the form of ointment, cream, oil, soap and shampoo contain non aqueous herbal extract of Wrightia Tinctoria, cocos nucifera, and suitable pharmaceutically/cosmetically accepted excipients for dermal use.

### CROSS-REFERENCE TO RELATED APPLICATIONS

The present application, filed under 35 U.S.C. 121, is a divisional application of U.S. non-provisional patent application Ser. No. 11/288,923 entitled “Composition for Safe and Effective Regression of Dermal Vessel Tortuosity,” filed on Nov. 28, 2005 and as such claims priority benefit under 35 U.S.C. 120 of this application. The disclosure of this application is incorporated herein by reference in its entirety.

### STATEMENT REGARDING FEDERALLY SPONSORED RESEARCH OR DEVELOPMENT

Not applicable.

### FIELD OF THE INVENTION

The present invention relates to compositions for Safe and Effective regression of Dermal Vessel Tortuosity. In particular, the present invention relates to compositions for safe and effective regression of Dermal Vessel Tortuosity in Psoriatic Lesions.

### BRIEF DESCRIPTION OF THE BACKGROUND ART

The epidermis of the skin is a non-vascularized layer of the skin. Different factors can result in increased blood vessel formation in the papillary dermis and these blood vessels may sometimes extend into the epidermis resulting in clinically significant skin manifestations. For example, over activated keratinocytes actively producing and secreting pro-angiogenic factors in the form of growth factors or cytokines can result in increased blood vessel formation in the papillary dermis which may sometime extend into the epidermis. Epidermal microvascular proliferation ultimately leads to epidermal keratinocyte hyperproliferation, thickening of the epidermis with parakeratosis of the stratum corneum and inflammatory infiltrate around the blood vessels in the papillary dermis [see FIG. 1]. The microvascular changes are also characterized by increased tortuosity of dermal capillary loops which precedes the development of epidermal hyperplasia. Histological studies, including immunocytochemistry, routine histology and electron microscopy have clearly established that alterations in the blood vessel formation of the skin discussed above are a prominent feature of psoriasis and there is a marked increase in cutaneous blood active edge of the psoriatic plaque [Braverman I M, Yen A. Ultrastructure of the capillary loops in the dermal papillae of psoriasis. J Invest Dermatol 1977: 68: 53-60].

Numerous therapies in the field of allopathy medicine [Treatment of psoriasis—Part 1—Topical Therapy and Phototherapy, Mark Lebwohl, MD, et al, American Academy of Dermatology—October 2001—Vol 45—November—4; Treatment of psoriasis—part 2—systemic Therapies, Mark Lebwohl, MD, et al—American Academy of Dermatology—November 2001—Vol 45—Number 5; The immunological basis for the treatment of psoriasis with new biological agents. James. G. krueger, M.D, American Academy of Dermatology—June 2002—Vol 46, Number 1, Pages 1-26; New psoriasis Treatments based upon a deeper understanding of the pathogenesis of psoriasis vulgaris and psoriatic arthritis —Jeffrey. P. callen et al American Academy of dermatology, August 2003—Vol 49, Number 5, Pages 351-356] have been researched and developed to reduce the Dermal Vessel Tortuosity especially related to Psoriasis. However, most of these therapies provide only temporary symptomatic relief and are either unsatisfactory or very expensive [National Psoriasis Conference, Boston Plaza Hotel, Aug. 5-8, 2005, Boston, Mass., USA.] and are associated with either short term or long term undesired side effect profiles. Herbal formulations are well known to minimize risk of undesired side effect profiles and hence provide a viable alternative therapy to manage this disease condition. Research efforts to develop Herbal formulations to treat this disease condition have been on the rise [Chopra, R. N., Nayar, S. C., and Chopra I. C., Glossary of Indian Medicinal Plants, C.S.I.R., P. 259, 1956; Murugesa Mudaliar, K. S., Gunapadam (Material Medica) Vegetable Section, Govt. of TamilNadu, P. 527 (1969); Venkatarajan, S., Sarabendra Vaithiya Muraigal, P. 160, 161 & 167 (1965); Wealth of India, Raw Materials, Vol. X, P. 588-590, CSIR., New Delhi (1976); Yugimuni Vaidya Chintamani (800) Stanza 494-518, B. Rathina Nayakar & Sons, Madras, India; Nair, C. P. R., Kurup, P. B., Pillai, K. G. B., Geetha, A., and Ramiah, N., Effect of Nimbidin in Psoriasis, Indian Medical Journal, October 1978] and there is a continuing need to develop Herbal formulations to treat this disease condition with minimal or no side effects. This patent provides Herbal formulations from Wrightia Tinctoria and Cocos Nucifera developed by a dermatologist and clinically proven to be safe and efficacious to reduce dermal tortuoisty.

### SUMMARY OF THE INVENTION

It is an object of the present invention to provide topical compositions for Safe and Effective regression of Dermal Vessel Tortuosity in the form of ointment, cream, oil, soap and shampoo containing non aqueous herbal extract and suitable pharmaceutically/cosmetically accepted excipients for dermal use.

This object and other objectives are provided by novel topical compositions for Safe and Effective regression of Dermal Vessel Tortuosity in the form of ointment, cream, oil, soap and shampoo which comprise non aqueous herbal extract of Wrightia Tinctoria, cocos nucifera, and suitable pharmaceutically/cosmetically accepted excipients for dermal use.

### BRIEF DESCRIPTION OF THE DRAWINGS

FIG. 1: Illustration of epidermal microvascular proliferation

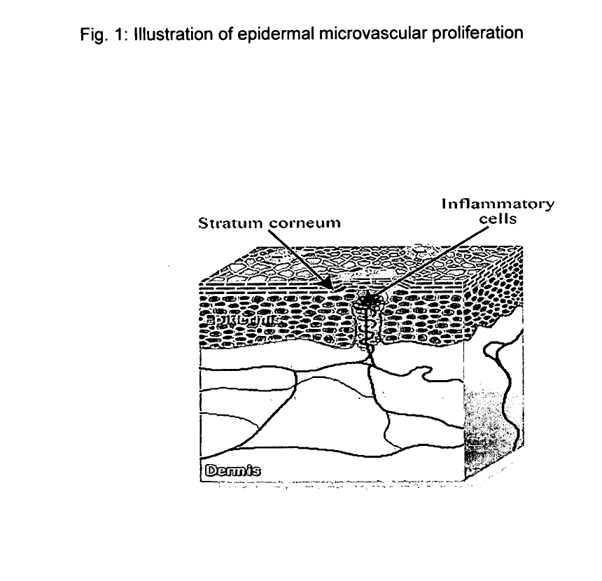

FIG. 2: Micro Graphs of Dermal Vessel Tortuosity—Before and after Treatment

### DETAILED DESCRIPTION OF THE INVENTION

The present invention relates to novel Herbal formulations which unexpectedly provide statistically superior efficacy to allopathy control formulations in reduction of dermal vessel tortuosity and is proven safe to use. The Novel Herbal formulations for the Safe and Effective regression of dermal vessel tortuosity are designed specifically for topical use in the form of ointment, shampoo, oil and soap. These compositions typically contain non-aqueous Herbal extract of Wrightia Tinctoria and Herbal extract of Cocos nucifera and pharmaceutically or cosmetically accepted excipients for dermal use in ointment, oil, soap and shampoo formulations.

The non aqueous medium of the present invention for Herbal extract is non-volatile oil, wherein non-volatile oil is preferably vegetable oil such as coconut oil, gingely oil, sunflower oil, corn oil, or refined vegetable oil. The non-volatile oil in the extract of the present invention generally comprises from about 80% to 99% weight percent of the extract.

The Herbal extract in the topical composition for Safe and Effective regression of Dermal Vessel Tortuosity is derived from Wrightia Tinctoria and is from either or combination of leaves, leafy stems and other cut portions of Wrightia Tinctoria plant. It is an apocynaceae tree growing throughout India. Its flowers are white and fragrant. Non aqueous extracts of Wrightia Tinctoria are prepared at ambient temperature and compounded with the other ingredients mentioned herein to prepare the different topical formulations for Ointment, Oil, Liquid soap and Shampoo. Other Herbal extracts in the formulation may include Melia Azardirachta Linn oils documented to have beneficial skin effects [Nair et al., 1978]. The topical composition of the present invention for Safe and Effective regression of Dermal Vessel Tortuosity generally comprises of extract of active Herbal ingredient mentioned above in the extraction medium in the amount from 1% to 20% weight percent.

The Herbal extract of Cocos Nucifera in the topical composition for Safe and Effective regression of Dermal Vessel Tortuosity is derived from the copra of the coconut. Copra of the coconut is dried and processed to extract oil which is purified and stabilized. The topical composition of the present invention for Safe and Effective regression of Dermal Vessel Tortuosity generally comprises of Herbal extract of cocos nucifera from the copra of the coconut present in the amount of 40% to 80%.

The topical ointment composition for Safe and Effective regression of Dermal Vessel Tortuosity described above include pharmaceutically accepted excipients such as Bees Wax, Paraffin (liquid, soft and hard), and other standard ointment bases or equivalents to optimize use characteristics (such as consistency, spreadability, . . . ) manufacturability and stability. The topical ointment composition of the present invention for Safe and Effective regression of Dermal Vessel Tortuosity generally comprises of excipients such as Bees Wax present in the amount of 1 to 5%, Paraffin present in the amount of 5 to 40% and/or standard ointment bases present in the amount of 5 to 50%.

The topical oil composition for Safe and Effective regression of Dermal Vessel Tortuosity described above include pharmaceutically accepted excipients such as Vegetable oil, animal oil, and synthetic oils such as mineral oil and liquid paraffin or equivalents to optimize use characteristics (such as consistency, spreadability, . . . ) manufacturability and stability. The topical oil composition of the present invention for Safe and Effective regression of Dermal Vessel Tortuosity generally comprises of excipients such as coconut oil present in the amount of 70 to 95%.

The topical liquid soap composition for Safe and Effective regression of Dermal Vessel Tortuosity described above include pharmaceutically accepted excipients such as water, surface active agents, thickeners or viscosity enhancers, foam boosters, and stabilizers or equivalents to optimize use characteristics (such as consistency, cleaning, spreadability, foaming, . . . ) manufacturability and stability. The topical liquid soap composition of the present invention for Safe and Effective regression of Dermal Vessel Tortuosity generally comprises of excipients such as water present in the amount of 60 to 85%, surface active agents present in the amount of 5 to 40%, thickeners or viscosity enhancers present in the amount of 0.5 to 8%, foam boosters present in the amount of 1 to 4% and stabilizers present in the amount of 0.5 to 2%.

The topical shampoo composition for Safe and Effective regression of Dermal Vessel Tortuosity described above include pharmaceutically accepted excipients such as water, surface active agents, thickeners or viscosity enhancers, foam boosters, and stabilizers or equivalents to optimize use characteristics (such as consistency, cleaning, spreadability, foaming, . . . ) manufacturability and stability. The topical shampoo composition of the present invention for Safe and Effective regression of Dermal Vessel Tortuosity generally comprises of excipients such as water present in the amount of 50 to 85%, surface active agents present in the amount of 10 to 30%, thickeners or viscosity enhancers present in the amount of 2 to 8%, foam boosters present in the amount of 2 to 6% and stabilizers present in the amount of 0.5 to 2%.

In addition, the topical composition for Safe and Effective regression of Dermal Vessel Tortuosity described above wherein Pharmaceutically or cosmetically accepted excipients in ointment, oil, liquid soap and shampoo formulations may include preservatives, coloring agents and fragrances as needed wherein preservatives, coloring agents and fragrances in ointment, oil, liquid soap and shampoo formulations is present in the amount of 0-5 total weight %.

The novel Herbal topical composition of the present invention described above containing non-aqueous Herbal extract of Wrightia Tinctoria and Herbal extract of Cocos nucifera and pharmaceutically or cosmetically accepted excipients for dermal use for Safe and Effective regression of Dermal Vessel Tortuosity will now be illustrated by the following example.

### EXAMPLE

Twenty patients were enrolled in a clinical study and were divided into two groups of 10 patients each. Group I was treated with the Herbal formulation (see Table 1 for details) once daily and Group II was treated with Allopathy control formulation (see Table 2 for details) once daily. All patients recruited were screened to be suffering from Dermal Vessel Tortuosity problems (Psoriasis patients).

TABLE 1
Herbal Ointment Formula

| No | Ingredient | Quantity |
|---|---|---|
| 1 | Wrightia Tinctoria | 5% |
| 2 | Cocos Nucifera | 65% |
| 3 | Bees Wax | 6% |
| 4 | Liquid Paraffin | 24% |
| 5 | Coloring Agent | QS |
| 6 | Fragrance | QS |

TABLE 2
Dithranol Ointment
(Allopathy Control)

| No | Ingredient | Quantity |
|---|---|---|
| 1 | Dithranol | 1% |
| 2 | Standard Ointment Base | QS |

Randomization was done as per standard statistical methods to minimize bias in the study. Patients were enrolled into the study on a first come first served basis and assigned a subject number sequentially. The assignment of each patient to the treatment group was determined by the randomization list provided by the statistician.

Each patient enrolled in the study voluntarily and received the treatment for 8 weeks. Skin Biopsies at the treatment site was taken from all patients at the beginning (T0) and end of the study (T8w) for Histopathological evaluation. In addition, at the beginning (T0), end of treatment (T8w) Haemogram analysis, Liver Function Testing and Renal Function Testing were done to document the safety profile of the treatments administered.

Histopathology of the skin biopsy was done by an expert pathologist and the Dermal Vessels Tortuosity parameter was measured at visits T₀ and T_{8W}. The results of the Dermal Vessels Tortuosity measurements were scored as follows: (+)=3 representing significant tortuosity of the dermal vessels; (±)=2 representing moderate tortuosity of the dermal vessels; and (−)=1 representing no tortuosity of the dermal vessels. Dermal Vessels Tortuosity parameter represents the degree of tortuosity of the dermal vessels. More active the disease more the tortuosity of the dermal vessels.

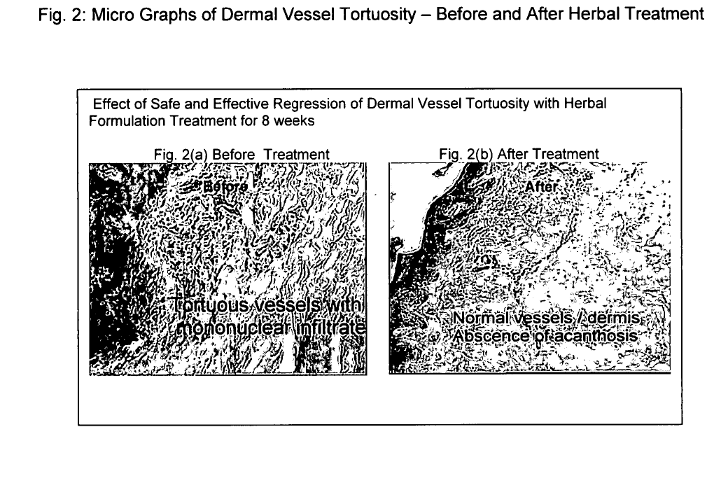

FIG. 2 presents photos of micrographs observed before and after treatment with patients treated for 8 weeks with the Herbal formulation presented in this invention.

It is clear from the photographs that the treatment with The Herbal formulation is very effective in regressing the dermal vessel tortuosities and clearing of the dermal infiltrate as compared to prior to the start of treatment.

Results of the statistical analysis of the Dermal Vessels Tortuosity measurement data for the 2 different groups of treatment are presented below in Table 3. A p-value of 0.05 is considered to be significant.

TABLE 3
Statistical Analysis of Histopathology Measurements for Dermal
Vessels Tortuosity

| | | | Allopathy | | | |
|---|---|---|---|---|---|---|
| | Herbal | | Control | | | |
| | (Group I) | | (Group II) | | | |
| | Mean | SD | Mean | SD | t | P-Value |
| Tow | 3 | 0 | 2.70 | 0.67 | 1.406 | 0.177 |
| T1w | 1.70 | .67 | 1.90 | 0.99 | 0.526 | 0.605 |
| Paired t | 6.091 | | 1.922 | | | |
| Statistic | | | | | | |
| Sig | 0.001 | | 0.087 | | | |
| (2 tailed) | | | | | | |

To examine the treatment effects, t-test was done with data between the two groups at the beginning and end of treatment. No statistical significance was observed (p>0.05) for treatment effects on the Dermal Vessels Tortuosity measurements at the beginning (p=0.177) and the end of treatment (p=0.605).

To examine the time effects within each group, paired t-test was done with data at the beginning and end of treatment within each group. With the Herbal Group, there was a statistically significant time effect (p-values equal to 0.001) on the Dermal Vessels Tortuosity measurements and it was found that the Dermal Vessels Tortuosity values decreased with time suggesting positive response to Herbal treatment with time.

However, with the Allopathy Control (Group II), it was found that there was no statistically significant time effect for the Allopathy control formulation (p-value equal to 0.087).

The statistical data analysis clearly indicates that the Herbal treatment for regression of dermal vessel tortuoisty is very safe and Effective and is superior to the allopathy control formulation.

The safety of the use of the Herbal Formulation of the present invention for regression of Dermal Vessel Tortuosity over the treatment period was evaluated by measuring Vital signs, Haemogram measurements, Liver function Test (LFT) measurements, and Renal Function Test (RFT) measurements and analyzing the data as a function of time. Vital signs were measured 6 times during treatment (T0, T1w, T2w, T4w, T6w, and T8w); Haemogram, LFT and RFT measurements were made only at the beginning and end of the treatment (T0, T8w).

Results of the Statistical analysis of the Vital Sign measurements (Systolic and Diastolic BP, pulse rate and respiratory measurements) are presented in Table 4. BP was measured using a manual mercury sphygmomometer and the unit of measurement is mm of Hg. Pulse rate was measured (beats per minute) in the radial artery by palpating the artery with the middle, index and ring finger. Respiratory rate was measured by watching the expansion of abdomen with each respiration and counting them for one minute.

TABLE 4
Statistical Analysis of Vital Sign Measurements for Herbal treatment.

| | | | | | | | Respiratory | |
|---|---|---|---|---|---|---|---|---|
| Time | BP-Systolic | | BP-Diastolic | | Pulse Rate | | Rate | |
| Points | Mean | SD | Mean | SD | Mean | SD | Mean | SD |
| 0w | 121.10 | 15.31 | 81.00 | 8.76 | 87.60 | 17.33 | 23.00 | 6.20 |
| T1w | 111.40 | 11.43 | 77.00 | 8.01 | 75.80 | 8.77 | 21.40 | 7.00 |
| T2w | 114.00 | 14.30 | 79.20 | 9.85 | 74.60 | 11.70 | 22.30 | 6.93 |
| T4w | 107.00 | 8.23 | 79.00 | 5.68 | 85.40 | 11.47 | 24.20 | 5.45 |
| T6w | 111.40 | 8.00 | 78.80 | 5.27 | 78.70 | 22.60 | 24.00 | 3.62 |
| T8w | 109.00 | 12.87 | 78.00 | 6.32 | 82.40 | 11.96 | 25.40 | 4.99 |
| Grand | 112.32 | 12.36 | 78.83 | 7.28 | 80.75 | 14.88 | 23.38 | 5.72 |
| Mean | | | | | | | | |

| | | | | |
|---|---|---|---|---|
| 1-Way | 1.674 | 0.317 | 1.273 | 0.612 |
| ANOVA | | | | |
| F-value | | | | |
| p-value | 0.157 | 0.901 | 0.289 | 0.691 |

A regular one-way ANOVA was also used to analyze the data at different time points for the different Vital signs measurements done. The data clearly indicates that there were no statistically significant time effects on BP Systolic measurements (p=0.157); BP Diastolic measurements (p=0.901); Pulse rate measurements (p=0.289) and Respiratory rate measurements (0.691) with the Herbal treatment. In summary, there is no statistically significant change in Vital Sign measurements with time due to treatment with the Herbal formulation of the present invention for Safe and Effective regression of dermal vessel tortuosity suggesting no safety issues.

Results of the Statistical analysis of the Haemogram measurements [Total count of White blood cells (TC), Differential white blood cells count as Polymorphonuclear neutrophil (DC-P), Lymphocytes (DC-L), Eosinophils (DC-E) and Haemoglobin (Hb)] are presented in Table 5. TC (Total count of White blood cells in the blood) was measured using Neubauer Counting Chamber and the normal range for TC measurements is 4000-11,000 cells per cubicmillimetre. DC-P, which stands for the percentage of P-Polymorphonuclear neutrophil, was measured using Neubauer Counting Chamber and the normal range for DC-P measurements is 55-65% of Total White Cell count. DC-L, which is the percentage of Lymphocytes present, was measured using Neubauer Counting chamber and the normal range for DC-L Measurements is 30-40% of the total white cell count. DC-L was measured. DC-E, which is the percentage of Eosinophils, was measured using the Neubauer Counting Chamber and the normal range for DC-E measurements is 1-7% of the total white blood cell count. DC-E was measured. HB which is Haemoglobin measurements was measured using RA 50 Biochemical analyzer and the normal range is 12-14 gms.

TABLE 5
Statistical Analysis of Haemogram Measurements for Herbal treatment.

| Time | TC | | DC-P | | DC-L | | DC-E | | HB | |
|---|---|---|---|---|---|---|---|---|---|---|
| Points | Mean | SD | Mean | SD | Mean | SD | Mean | SD | Mean | SD |
| T0w | 7343 | 1589 | 57.30 | 2.95 | 37.90 | 1.79 | 4.80 | 2.90 | 13.02 | 1.72 |
| T8w | 8634 | 1105 | 58.90 | 2.69 | 37.10 | 2.38 | 4.00 | 2.49 | 12.95 | 0.94 |
| Paired in | −1291 | 2279 | −1.60 | 3.78 | 0.80 | 3.22 | 0.80 | 3.77 | 0.075 | 2.13 |
| differ mean | | | | | | | | | | |

| | | | | | |
|---|---|---|---|---|---|
| Paired t | −1.791 | −1.340 | 0.784 | 0.672 | 0.100 |
| statistic | | | | | |
| Sig | 0.107 | 0.213 | 0.453 | 0.519 | 0.924 |
| (2-tailed) | | | | | |

To examine the time effects paired t-test was done with data at the beginning and end of treatment for the different Haemogram measurements done. The data clearly indicates that there were no statistically significant time effects on TC measurements (p=0.107); DC-P measurements (p=0.213); DC-L measurements (p=0.453); DC-E measurements (p=0.519) and HB measurements (p=0.924) with the Herbal treatment. In summary, there is no statistically significant change in Haemogram measurements with time due to treatment with the Herbal formulation of the present invention for Safe and Effective regression of dermal vessel tortuosity suggesting no safety issues.

Results of the Statistical analysis of the Liver Function Test (LFT) measurements [Serum Glutamic Oxalo acetic Transaminase (SGOT), Serum Glutamic Pyruvic Transaminase (SGPT) and Serum Bilirubin] are presented in Table 6. SGOT, serum glutamic—oxalo acetic transaminase (international unit per liter), was measured at visits T₀ and T_{8W}. And the normal range is 0-46 I.U/L. SGPT, Serum glutamic pyruvic transaminase (international units/liter) was measured at visits T₀ and T_{8W}. And the normal SGPT ranges from 0 to 49 IU/L. Serum Bilirubin was measured at visits T₀ and T_{8W} and the normal Serum Bilirubin ranges from 0.0 to 1.0 mg/dl.

TABLE 6
Statistical Analysis of Liver Function Test (LFT)
Measurements for Herbal treatment.

| Time | SGOT | | SGPT | | Serum Bilirubin | |
|---|---|---|---|---|---|---|
| Points | Mean | SD | Mean | SD | Mean | SD |
| T0w | 24.90 | 8.80 | 26.10 | 14.78 | 0.73 | 0.23 |
| T8w | 24.00 | 8.94 | 26.60 | 11.01 | 0.69 | 0.24 |
| Paired in | 0.90 | 10.97 | −0.50 | 11.24 | 0.035 | 0.31 |
| differ | | | | | | |
| mean | | | | | | |

| | | | |
|---|---|---|---|
| Paired t | 0.259 | 0.141 | 0.352 |
| statistic | | | |
| Sig | 0.801 | 0.891 | 0.733 |
| (2-tailed) | | | |

To examine the time effects paired t-test was done with data at the beginning and end of treatment for the different LFT measurements done. The data clearly indicates that there were no statistically significant time effects on SGOT measurements (p=0.801); SGPT measurements (p=0.891); and Serum Bilirubin measurements (p=0.733) with the Herbal treatment. In summary, there is no statistically significant change in LFT measurements with time due to treatment with the Herbal formulation of the present invention for Safe and Effective regression of dermal vessel tortuosity suggesting no safety issues.

Results of the Statistical analysis of the Renal Function Test (RFT) measurements [Serum Creatinine and Serum Urea] are presented in Table 7.

Serum Creatinine was measured at visits T₀ and T_{8W}. And the normal Serum Creatinine value ranges from 0.8 to 1.4 mg/dl. Serum Urea was measured at visits T₀ and T_{8W}. And the normal Serum Urea value ranges from 10 to 50 mg/dl.

TABLE 7
Statistical Analysis of Renal Function
Test (RFT) Measurements for Herbal
treatment.

| | | |
|---|---|---|
| Time | Serum Creatinine | Serum Urea |

| Points | Mean | SD | Mean | SD |
|---|---|---|---|---|
| T0w | 1.06 | 0.22 | 32.40 | 17.50 |
| T8w | 1.08 | 0.18 | 25.49 | 7.75 |
| Paired in | −0.021 | 0.244 | 6.91 | 18.81 |
| differ | | | | |
| mean | | | | |

| | | |
|---|---|---|
| Paired t | −0.271 | 1.161 |
| statistic | | |
| Sig | 0.792 | 0.275 |
| (2-tailed) | | |

To examine the time effects paired t-test was done with data at the beginning and end of treatment for the different RFT measurements done. The data clearly indicates that there were no statistically significant time effects on Serum Creatinine measurements (p=0.792) and Serum Urea measurements (p=0.275) with the Herbal treatment. In summary, there is no statistically significant change in RFT measurements with time due to treatment with the Herbal formulation of the present invention for Safe and Effective regression of dermal vessel tortuosity suggesting no safety issues.

It is clear that the Histopathological examination and statistical analysis of the clinical data that the novel Herbal formulation described in the present invention is very effective in treatment of dermal vessel tortuosity and is superior to the allopathy control. In addition, evaluation of Haemogram, LFT and RFT test results clearly show that the Herbal formula of the present invention is also very safe to use on humans.

Other modifications and variations of the present invention will become apparent to those skilled in the art from an examination of the above specification and examples. Therefore, other variations of the present invention may be made which fall within the scope of the appended claims even though such variations were not specifically discussed above.

I claim:

1. A topical composition for safe and effective regression of Dermal Vessel Tortuosity in Psoriatic Lesions, comprising:
Non-aqueous Herbal extract of Wrightia Tinctoria, and
Herbal extract of Cocos nucifera and
Pharmaceutically or cosmetically accepted excipients for dermal use in ointment, oil, soap and shampoo formulations.

2. The topical composition for safe and effective regression of Dermal Vessel Tortuosity in Psoriatic Lesions according to claim 1, wherein non aqueous medium for Herbal extract is non-volatile oil.

3. The topical composition for safe and effective regression of Dermal Vessel Tortuosity in Psoriatic Lesions according to claim 2, wherein non-volatile oil is preferably vegetable oil such as coconut oil, gingely oil, sunflower oil, corn oil, or refined vegetable oil.

4. The topical composition for safe and effective regression of Dermal Vessel Tortuosity in Psoriatic Lesions according to claim 2, wherein non-volatile oil is present in the extract in the amount from 80% to 99% weight percent of the extract.

5. The topical composition for safe and effective regression of Dermal Vessel Tortuosity in Psoriatic Lesions according to claim 1, wherein Herbal extract of Wrightia Tinctoria is from either or combination of leaves, leafy stems and other cut portions of Wrightia Tinctoria plant.

6. The topical composition for safe and effective regression of Dermal Vessel Tortuosity in Psoriatic Lesions according to claim 5, wherein Herbal extract of Wrightia Tinctoria is present in the extraction medium in the amount from 1% to 20% weight percent.

7. The topical composition for safe and effective regression of Dermal Vessel Tortuosity in Psoriatic Lesions according to claim 1, wherein Herbal extract of cocos nucifera is from the copra of the coconut.

8. The topical composition for safe and effective regression of Dermal Vessel Tortuosity in Psoriatic Lesions according to claim 7, wherein Herbal extract of cocos nucifera from the copra of the coconut is present in the amount of 40% to 80%.

9. The topical composition for safe and effective regression of Dermal Vessel Tortuosity in Psoriatic Lesions according to claim 1, wherein Pharmaceutically accepted excipients in ointment formulations include Bees Wax, Paraffin (liquid, soft and hard), and other standard ointment bases.

10. The topical composition for safe and effective regression of Dermal Vessel Tortuosity in Psoriatic Lesions according to claim 9, wherein in ointment formulations the Beeswax is present in the amount of 1 to 5%.

11. The topical composition for safe and effective regression of Dermal Vessel Tortuosity in Psoriatic Lesions according to claim 9, wherein in ointment formulations the Paraffin is present in the amount of 5 to 40%.

12. The topical composition for safe and effective regression of Dermal Vessel Tortuosity in Psoriatic Lesions according to claim 1, wherein Pharmaceutically accepted excipients in oil formulations include Vegetable oil, animal oil, and synthetic oils such as mineral oil and liquid paraffin.

13. The topical composition for safe and effective regression of Dermal Vessel Tortuosity in Psoriatic Lesions according to claim 12, wherein in oil formulations vegetable oil, preferably coconut oil is present in the amount of 70 to 95%.

14. The topical composition for safe and effective regression of Dermal Vessel Tortuosity in Psoriatic Lesions according to claim 1, wherein cosmetically accepted excipients in liquid soap formulations include water, surface active agents, thickeners or viscosity enhancers, foam boosters, and stabilizers.

15. The topical composition for safe and effective regression of Dermal Vessel Tortuosity in Psoriatic Lesions according to claim 14, wherein in liquid soap formulations water is present in the amount of 60 to 85%.

16. The topical composition for safe and effective regression of Dermal Vessel Tortuosity in Psoriatic Lesions according to claim 14, wherein in liquid soap formulations surface active agents is present in the amount of 5 to 40%.

17. The topical composition for safe and effective regression of Dermal Vessel Tortuosity in Psoriatic Lesions according to claim 14, wherein in liquid soap formulations thickeners or viscosity enhancers is present in the amount of 0.5 to 8%.

18. The topical composition for safe and effective regression of Dermal Vessel Tortuosity in Psoriatic Lesions according to claim 14, wherein in liquid soap formulations foam boosters is present in the amount of 1 to 4%.

19. The topical composition for safe and effective regression of Dermal Vessel Tortuosity in Psoriatic Lesions according to claim 14, wherein in liquid soap formulations stabilizers is present in the amount of 0.5 to 2%.

20. The topical composition for safe and effective regression of Dermal Vessel Tortuosity in Psoriatic Lesions according to claim 1, wherein cosmetically accepted excipients in shampoo formulations include water, surface active agents, thickeners or viscosity enhancers, foam boosters, and stabilizers.

21. The topical composition for safe and effective regression of Dermal Vessel Tortuosity in Psoriatic Lesions according to claim 20, wherein in shampoo formulations water is present in the amount of 50 to 85%.

22. The topical composition for safe and effective regression of Dermal Vessel Tortuosity in Psoriatic Lesions according to claim 20, wherein in shampoo formulations surface active agents is present in the amount of 10 to 30%.

23. The topical composition for safe and effective regression of Dermal Vessel Tortuosity in Psoriatic Lesions according to claim 20, wherein in shampoo formulations thickeners or viscosity enhancers is present in the amount of 0.5 to 8%.

24. The topical composition for safe and effective regression of Dermal Vessel Tortuosity in Psoriatic Lesions according to claim 20, wherein in shampoo formulations foam boosters is present in the amount of 2 to 6%.

25. The topical composition for safe and effective regression of Dermal Vessel Tortuosity in Psoriatic Lesions according to claim 20, wherein in shampoo formulations stabilizers is present in the amount of 0.5 to 2.0%.

26. The topical composition for safe and effective regression of Dermal Vessel Tortuosity in Psoriatic Lesions according to claim 1, wherein pharmaceutically or cosmetically accepted excipients in ointment, oil, and liquid soap and shampoo formulations include preservatives, coloring agents and fragrances as needed.

27. The topical composition for safe and effective regression of Dermal Vessel Tortuosity in Psoriatic Lesions according to claim 26, wherein preservatives, coloring agents and fragrances in ointment, oil, liquid soap and shampoo formulations is present in the amount of 0-5 total weight %.
